(19) United States
(12) Patent Application Publication
(10) Pub. No.: US 2025/0270615 A1
(43) Pub. Date: Aug. 28, 2025

(54) METHOD FOR DETECTING THE MICROBIAL GROWTH POTENTIAL OF AN ANTISCALANT COMPOSITION IN NATURAL SEAWATER

(71) Applicants: ACWA POWER Company, Riyadh (SA); King Abdullah University of Science and Technology, Thuwal (SA)

(72) Inventors: Graciela Gonzalez GIL, Panama (PA); Johannes Simon VROUWENVELDER, Nieuwegein (NL); Ratul DAS, Thuwal (SA); Thomas ALTMANN, Dubai (AE)

(21) Appl. No.: 18/584,973

(22) Filed: Feb. 22, 2024

(51) Int. Cl. C12Q 1/18; C12N 1/20; C12Q 1/06; G01N 1/40; G01N 15/14

(52) U.S. Cl. CPC C12Q 1/18; C12N 1/20; C12Q 1/06; G01N 1/4077; G01N 15/14; G01N 2001/4088; G01N 2015/1486

(57) ABSTRACT

The present disclosure provides a method of determining the microbial growth potential of at least one antiscalant composition in natural seawater comprising at least the following steps; filtering natural seawater to obtain seawater comprising autochthonous bacteria having a predetermined bacterial cell concentration, adding at least one antiscalant composition comprising at least one antiscalant to the seawater to obtain a mixture, wherein the concentration of the at least one antiscalant is in the range of 0.1 to 100 mg/L, incubating the mixture; and determining the bacterial cell concentration of the mixture at regular intervals to determine the microbial growth potential of the at least one antiscalant composition in the natural seawater.

### TECHNOLOGICAL FIELD

The present disclosure relates to a method for detecting the microbial growth potential of an antiscalant composition in natural seawater.

### BACKGROUND

In arid or water-stress regions, seawater desalination is one of the methods used for obtaining fresh water. Seawater can be desalinated by reverse osmosis (RO) technique. However, the accumulation of unwanted material on the RO membrane surface, collectively called fouling, is a major challenge in improving efficiency of the RO desalination technique for future sustainable freshwater supplies. Fouling and biofouling leads to an increased energy requirement for water production, decreased membrane efficiency, and ultimately leads to an early membrane failure.

To mitigate this problem, various antiscalants have been developed. Antiscalants are chemicals that inhibit the formation of minerals through chelation, dispersion, and/or by blocking crystal growth sites. The addition of antiscalants during pretreatment of the feed water is very cost-effective and convenient method for membrane scaling control at high water recoveries. Current commercial antiscalants are mainly based on phosphonates, or synthetic organic polymers or co-polymers.

However, the antiscalants differ in their effectiveness and hence evaluating the bacterial growth potential of commercially available antiscalants is essential for a rational selection of these chemicals. Although antiscalants mitigate inorganic fouling, they can increase the potential of biofouling, which is regarded as the most difficult fouling problem to deal with. Depending on their chemical components, antiscalants can function as phosphorus and or carbon sources for microbial growth. Consequently, evaluating the bacterial growth potential of the commercially available antiscalants is essential for a rational selection of these chemicals.

However, the methods disclosed in the prior art have limitations. Previous evaluations on the bacterial growth potential of antiscalants substantially helped realize that the prevention of a scale problem could translate into a RO feed spacer and membrane biofouling problem (Aranjo et al., 2012; Vrouwenvelder et al., 2009). However, those evaluations suffer from several limitations. Among others, previous antiscalant growth potential studies were conducted using drinking water inoculated with two single bacterial species isolated from freshwater (Vrouwenvelder et al., 2000) or seawater inoculated with a model bacterial species (Sweity et al., 2015). These conditions, however, are in stark contrast to those in natural seawater with diverse bacterial populations. Furthermore, previous studies provide no or limited information regarding the chemical composition of the tested antiscalants, which is relevant to understanding the correlation between chemical structure of different antiscalants and their potential to promote bacterial growth.

Moreover, the number of commercially available antiscalants for RO systems is large, new antiscalants are constantly introduced in the market, and providers do not disclose the antiscalants' chemical composition. Testing all available antiscalants for their growth potential is not practical.

Therefore, there is a need to determine the bacterial growth potential of antiscalants under relevant natural seawater conditions, and ideally, for predictive purposes, relate the antiscalants biological response to their chemical composition.

Therefore, an object of the present invention is to provide a method of detecting the microbial growth potential of an antiscalant composition in natural seawater. Specifically, wherein the seawater comprises autochthonous seawater bacteria. Further, it is also an object to establish a relationship between the microbial growth potential of an antiscalant composition in seawater and the chemical characteristics of the antiscalant such as its carbon content, phosphorous content and structural features as determined by NMR spectra.

It was found that the present invention provides a method that is more reliable for detecting the microbial growth potential of an antiscalant composition in natural seawater. Further, the method provides a correlation between the chemical structural features of antiscalants and their microbial growth potential in natural seawater.

### BRIEF SUMMARY

Accordingly, an aspect of the present invention is a method for determining the microbial growth potential of at least one antiscalant composition in natural seawater comprising at least the following steps: filtering natural seawater to obtain seawater comprising bacteria having a predetermined bacterial cell concentration; adding at least one antiscalant composition comprising at least one antiscalant to the seawater to obtain a mixture, wherein the concentration of the at least one antiscalant is in the range of 0.1 to 100 mg/L; and incubating the mixture; and determining the bacterial cell concentration of the mixture at regular intervals to determine the microbial growth potential of the at least one antiscalant composition in the natural seawater.

In one embodiment, the method further comprises a step of determining the chemical structure of the at least one antiscalant present in the at least one antiscalant composition.

In another embodiment, the chemical structure of the at least one antiscalant is determined based on at least one technique selected from ¹H, ¹³C, and ³¹P NMR spectra.

### BRIEF DESCRIPTION OF DRAWINGS

Having thus described example embodiments of the disclosure in general terms, reference will now be made to the accompanying drawings, which are not necessarily drawn to scale, and wherein:

FIG. 1A illustrates a graph showing the total organic carbon (TOC) of antiscalants (AS);

FIG. 1B illustrates a graph showing the phosphorous content of antiscalants (AS);

FIG. 1C illustrates antiscalants' NMR spectra showing chemical shifts of proton (¹H), carbon (¹³C) and phosphorus (³¹P);

FIG. 1D illustrates a Dendrogram showing the clustering of antiscalants based on concatenated NMR spectra;

FIG. 2A illustrates a graph showing the bacterial growth potential of different antiscalants (AS) in Red Sea water;

FIG. 2B illustrates a table showing the bacterial growth from antiscalant expressed as assimilable organic carbon (AOC) was calculated assuming a yield of 4.6×10⁶ cells μg C⁻¹.

### DETAILED DESCRIPTION

In the following description, for purposes of explanation, numerous specific details are set forth in order to provide a thorough understanding of the present disclosure. It will be apparent, however, to one skilled in the art that the present disclosure may be practiced without these specific details. In other instances, well-known processes, well-known apparatus structures, and well-known techniques may not be described in detail.

The terminology used, in the present disclosure, is only for the purpose of explaining a particular embodiment and such terminology shall not be considered to limit the scope of the present disclosure. As used in the present disclosure, the forms “a,” “an,” and “the” may be intended to include the plural forms as well, unless the context clearly suggests otherwise. The terms “comprises,” “comprising,” “including,” and “having,” are open ended transitional phrases and therefore specify the presence of stated features, integers, steps, operations, elements, modules, units and/or components, but do not forbid the presence or addition of one or more other features, integers, steps, operations, elements, components, and/or groups thereof. The particular order of steps disclosed in the method and process of the present disclosure is not to be construed as necessarily requiring their performance as described or illustrated. It is also to be understood that additional or alternative steps may be employed.

The terms first, second, third, etc., should not be construed to limit the scope of the present disclosure as the aforementioned terms may be only used to distinguish one element, component, region, layer or section from another component, region, layer or section. Terms such as first, second, third etc., when used herein do not imply a specific sequence or order unless clearly suggested by the present disclosure.

The method of the present invention is carried out in natural seawater as a medium. Preferably seawater having a predetermined bacterial population has been used. An autochthonous bacterial population (from the same natural seawater sample) is the inoculum in the process of the present invention.

The bacterial growth was assessed by a high-throughput single-bacterial cell detection method (flow cytometry, FCM).

Chemical structural features of antiscalants were determined by nuclear magnetic resonance (NMR) scans. The antiscalants were classified according to chemical structural features. This is especially important since the tested commercial antiscalant compositions did not disclose the chemical identity of the antiscalant present in the composition. A correlation was derived between the structural features of antiscalants and their microbial growth potential in seawater.

The NMR scans enabled sensitive antiscalant fingerprinting which allows an informed selection of antiscalants and contributes to biofouling control in seawater desalination plants.

The present disclosure provides a reliable process for detecting the microbial growth potential of an antiscalant composition in natural seawater.

In an aspect, the present invention provides a method of detecting the microbial growth potential of at least one antiscalant composition in natural seawater. The method comprises the following steps: filtering natural seawater to obtain seawater comprising bacteria having a predetermined bacterial cell concentration; adding at least one antiscalant composition comprising at least one antiscalant to the seawater to obtain a mixture, wherein the concentration of the at least one antiscalant in the mixture is in the range of 0.1 to 100 mg/L; and incubating the mixture, and determining the bacterial cell concentration of the mixture at regular intervals to determine the microbial growth potential of the at least one antiscalant composition in the natural seawater.

In one embodiment, the natural seawater may be processed to obtain the seawater having a predetermined bacterial cell concentration that is suitable for the method of the present invention.

The method for determining the microbial growth potential of at least one antiscalant composition in seawater is described in detail herein below.

In a first step, seawater comprising bacteria having a predetermined bacterial cell concentration is obtained by filtering natural seawater.

In accordance with the embodiments of the present disclosure, the natural seawater is seawater from any seawater source.

In an embodiment of the present disclosure, the natural seawater is obtained from Red Sea.

In an embodiment of the present disclosure, the bacteria are autochthonous seawater bacteria.

In accordance with the embodiments of the present disclosure, the predetermined bacterial cell concentration is in the range of 5000 to 50000 bacterial cells mL⁻¹.

In accordance with the embodiments of the present disclosure, the predetermined bacterial cell concentration is in the range of 7000 to 40000 bacterial cells mL⁻¹.

In accordance with the embodiments of the present disclosure, the predetermined bacterial cell concentration is in the range of 10000 to 30000 bacterial cells mL⁻¹.

In an exemplary embodiment of the present disclosure, the predetermined bacterial cell concentration is 21000±1200 bacterial cells mL⁻¹.

The natural seawater has a high microbial content. The natural seawater is serially filtered to obtain seawater comprising bacteria having a predetermined bacterial cell concentration.

The filtration is carried out using known techniques.

In accordance with an exemplary embodiment of the present disclosure, the filtration is carried out through pre-cleaned 0.45 μm pore size and 0.2 μm pore size filters.

The process of filtration allowed removing protozoa to avoid bacterial grazing, which could result in a large underestimation of a sample's bacterial growth potential.

In a second step, at least one antiscalant composition comprising at least one antiscalant is added to the seawater to obtain a mixture, wherein the concentration of the at least one antiscalant is in the range of 0.1 to 100 mg/L.

In accordance with the embodiments of the present disclosure, at least one antiscalant is selected from phosphonate-based antiscalant, polymer-based antiscalant, and biopolymer-based antiscalant.

In accordance with the embodiments of the present disclosure, the at least one antiscalant is selected from amino trimethylene phosphonic acid, 1-hydroxyethylidene-(1,1-diphosphonic acid), hexamethylenediamine-tetra(methylenephosphonic acid), polyacrylate-maleic acid copolymer, and carboxy methyl inulin.

In accordance with the embodiments of the present disclosure, the concentration of the antiscalant in the mixture is in the range of 0.1 to 100 mg/L.

In accordance with an exemplary embodiment of the present disclosure, the concentration of the antiscalant in the mixture is 50 mg/L.

The concentration of the at least one antiscalant in the mixture at 50 mg antiscalant L⁻¹ is a representation of general working conditions. In general practice (i) the antiscalants dosing range varies widely (0.1 to 100 mg/L), (ii) this value is in the upper dosing range of the antiscalant, and (iii) the concentration of the antiscalant will increase along the RO membrane modules (arranged in series in the pressure vessel) as a result of solute retention leading to a higher concentration on the membrane surface than in the bulk liquid (i.e., concentration polarization).

In a third step, the mixture obtained in the second step is incubated.

In accordance with the embodiments of the present disclosure, the mixture is incubated at 20° C. to 40° C.

In accordance with an exemplary embodiment of the present disclosure, the mixture is incubated at 30° C.

In accordance with the embodiments of the present disclosure, the mixture is incubated in dark.

In accordance with the embodiments of the present disclosure, the mixture is incubated for 2 to 10 days.

In accordance with the embodiments of the present disclosure, the mixture is incubated for 5 to 10 days.

In accordance with an exemplary embodiment of the present disclosure, the mixture is incubated for 8 days.

The bacterial cell concentration of the mixture was determined at regular intervals to determine the microbial growth potential of the at least one antiscalant composition in the seawater.

In accordance with the embodiments of the present disclosure, the bacterial cell concentration of the mixture is determined at regular intervals during incubation.

In accordance with an exemplary embodiment of the present disclosure, the bacterial cell concentration of the mixture is determined at 24-hour intervals.

In accordance with the embodiments of the present disclosure, the bacterial cell concentration of the mixture is determined by flow cytometry.

The microbial growth in the mixture was evaluated by determining the change in bacterial cell concentration of the mixture over the incubation time.

For each time interval, the bacterial cell concentration of the incubated mixture was determined by calculating the difference between the bacterial cell concentrations of the sample and a control. The control is similar to the sample except that the control does not contain the antiscalant.

In accordance with the embodiments of the present disclosure, the method further comprises a step of determining the chemical structure of the at least one antiscalant present in the at least one antiscalant composition.

Chemical suppliers do not disclose the specific chemical formulation of antiscalants, which is relevant to understanding their growth potential.

A biodegradable antiscalant can provide carbon for bacterial growth. Therefore, the total organic content (TOC) of the antiscalants was measured.

The number of bacteria that grew in the presence of an antiscalant in excess to that of the control (without antiscalant) can be expressed in terms of carbon utilized from antiscalant to produce such number of bacteria. Comparing those values with the measured TOC in antiscalants may inform on the antiscalants' biodegradability.

It was observed that the polymeric antiscalants, and the HEDP-like phosphonate (AS-7) had a low biodegradability. All phosphonates ATMP-like antiscalants containing phosphate as contaminant had the potential to promote bacterial growth in excess to that of the control with no antiscalant.

Besides carbon, the phosphorous in the antiscalants can promote microbial growth. Therefore, the phosphorous content of the antiscalants was measured.

In accordance with the embodiments of the present disclosure, the chemical structure of the at least one antiscalant is determined based on at least one technique selected from ¹H, ¹³C, and ³¹P NMR spectra. In the ATMP-like antiscalants, ³¹P NMR revealed the presence of contaminant phosphate.

Since the chemical structure of antiscalants determines their growth potential, NMR-based chemical fingerprints of the antiscalants were analyzed.

Hierarchical cluster analysis allowed the grouping of the antiscalants based on their chemical NMR fingerprint.

Based on their chemical structure, the antiscalants are broadly classified as phosphorus-based and non-phosphorus-based. Systems containing organic carbon and none or insufficient bioavailable phosphorus for bacterial growth are regarded as phosphorus-limiting.

It was observed that the bacteria in the seawater at the time of the growth potential were phosphorus limited.

It was observed that not all phosphorus-based antiscalants promote bacterial growth, even at phosphorus-limiting conditions.

It was found that the grouping antiscalants through their NMR fingerprints is advantageous. There are a large number of antiscalants in the market, and for the majority, their chemical composition is undisclosed. This represents a limitation towards a rational selection for their application in RO systems since the chemical structure of antiscalants inherently determines their potential to promote or not bacterial growth (of course, besides their effectiveness as antiscalant).

With the help of an NMR fingerprint approach, it is possible to group antiscalants by their chemical signatures even if the identity of the antiscalant is unknown. NMR analysis yields unique spectral fingerprints for different molecules and functional groups.

A hierarchical analysis of antiscalants NMR spectra can reveal clusters allowing for approximating the chemical structure of their main components.

Thus, given a set of NMR spectra of chemically defined antiscalants and their clustering, it would be possible to classify commercial antiscalants for which no chemical information is provided.

It was observed that the bacterial growth potential of antiscalants is related to their chemical fingerprint. The bacterial growth potential of eight antiscalants was measured in natural seawater and with a seawater autochthonous bacterial community. The antiscalants' bacterial growth potential reflected their chemical NMR fingerprint.

It was observed that the phosphonate based antiscalants made of ATMP and HDTMP promoted bacterial growth.

It was observed that the antiscalant HEDP is a phosphonate that did not promote bacterial growth.

It was observed that the phosphorus-free antiscalants, i.e. a biopolymer and a synthetic polymer are less susceptible to promoting bacterial growth.

While the presently claimed invention has been described in terms of its specific embodiments, certain modifications and equivalents will be apparent to those skilled in the art and are intended to be included within the scope of the presently claimed invention.

The presently claimed invention is illustrated in detail by non-restrictive working examples which follow. More particularly, the test methods specified hereinafter are part of the general disclosure of the application and are not restricted to the specific working examples.

The following materials are used in the examples:

The eight commercial antiscalant samples were obtained from local desalination plants. These eight antiscalant samples were coded as AS 1 to AS 8.

The source of natural seawater was the Red Sea. Natural seawater from the Red Sea was collected in July 2021 from the intake of the seawater desalination pilot plant in KAUST, Saudi Arabia.

The collected natural seawater was stored in assimilable organic carbon-free (AOC) borosilicate glass bottles.

The collected natural seawater had an initial measured bacterial cell concentration of 200,000 cells mL⁻¹. The natural seawater was serially filtered to remove microbial cells and to set a desired low initial cell concentration for the growth potential assay within approximately 10,000 to 20,000 bacterial cells mL⁻¹. This seawater was used in the method of the present disclosure.

Natural seawater (1.0 L) was serially filtered through pre-cleaned 0.45 μm pore size and 0.2 μm pore size polyethersulfone filters (Satorius Stedium Minisart). The filters were replaced every 100 mL.

The commercial antiscalant composition was diluted with distilled water to obtain a stock solution having an antiscalant concentration of 3 g L⁻¹. Accordingly, 8 antiscalant stock solutions were prepared from 8 antiscalant compositions.

Antiscalants were added from the stock solutions (3 g L⁻¹) to the vials at a final concentration of 50 mg L⁻¹.

### Mixtures of Seawater and Antiscalant Composition:

30 mL of the resulting filtered seawater with a bacterial cell concentration of 21,000±1200 cells mL⁻¹ was added to AOC-free 40 mL borosilicate glass vials with screw caps containing PTFE-lined silicone septa.

Antiscalants from the 8 stock solutions (3 g L⁻¹) were added to 8 vials to obtain 8 mixtures comprising the antiscalant at a final concentration of 50 mg L⁻¹.

Vials with no antiscalant addition were included as a control.

The vials were incubated at 30° C. in the dark and bacterial growth was determined by measuring cell concentrations over time using flow cytometry. Each growth potential test was conducted in three independent biological replicates.

Flow cytometry (FCM) was used to measure the bacterial cell concentration in the vials at an interval of 24 h.

To this end, 5.0 μL SYBR Green I (Invitrogen, 100× diluted in 10 mM Tris buffer pH=8.1) were added to a final 0.5 mL vial samples in amber microfuge tubes followed by mixing and incubation for 10 min in the dark at 35° C.

Stained cells were measured with a BD Accuri C6 instrument in fixed 50 μL volume mode with a sample flow rate of 35 μL min⁻¹. The blue 488 nm laser (50 mW) was used to collect the green fluorescence at FL1=533 30 nm, the red fluorescence FL3>670 nm, as well as intrinsic cell parameters were given by the side scattered (SSH) and forward scattered (FSH) light signals. Electronic gating was used to separate positive microbial stained signals from instrument, and water background noise as described by Prest et al. (2013).

Filtered sterilized (0.1 μm) ultrapure water, as well as unstained samples, were used as controls in all runs.

The instrument performance was verified using Spherotech 8-peak validation beads. To evaluate whether bacterial growth on antiscalants was greater than the control, the bacterial growth curves were compared using a one-tailed paired Wilcoxon signed-rank test at a significance level α=0.05.

### Chemical Composition and Characterization of Antiscalants

The chemical characterization of the antiscalants AS 1 to AS 8 was carried out as follows.

### Example 1-8: Chemical Composition of Antiscalants—Carbon Content of Antiscalants

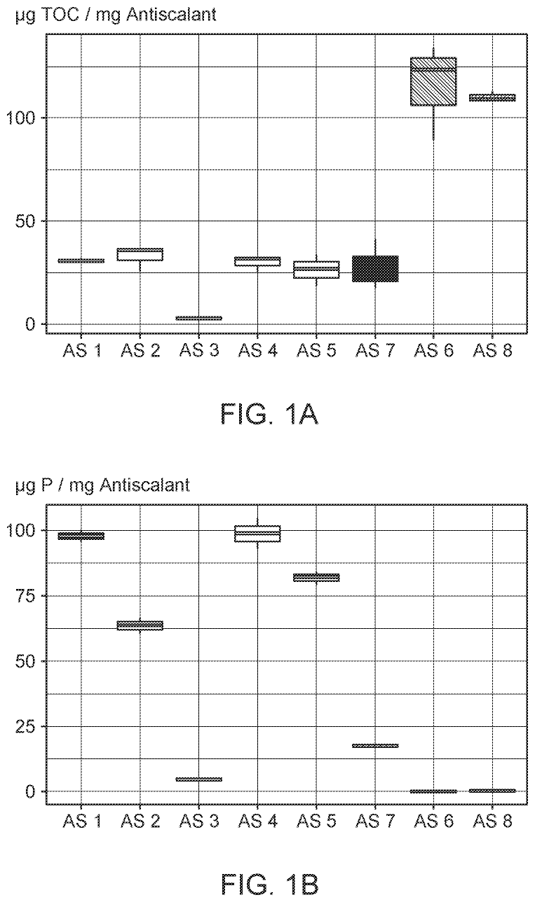

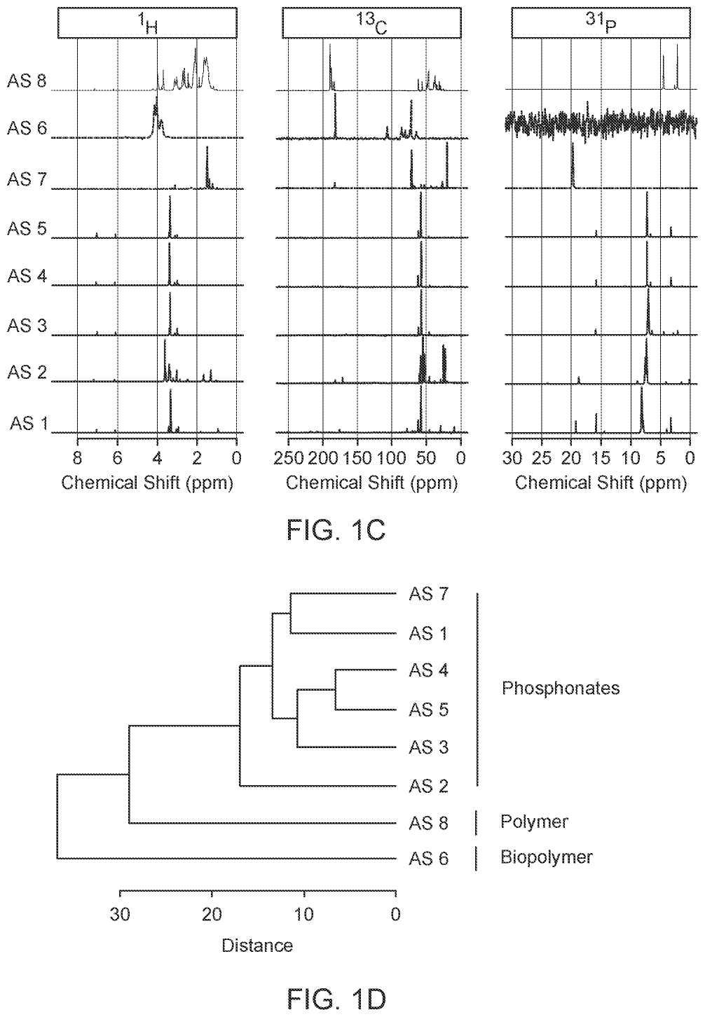

The results of TOC determination of antiscalants AS-1 to AS-8 are provided in FIG. 1A.

It was observed that AS-1 to AS-5 and AS-7 had low carbon contents, i.e. at levels <40 μg TOC mg antiscalant-1. Among them, AS 3 had the lowest carbon content (2.6±0.7 μg TOC mg antiscalant-1).

In contrast, the TOC in antiscalants AS 6 (115±23 μg TOC mg antiscalant-1) and AS 8 (110±2 μg TOC mg antiscalant-1) was significantly higher (p<0.05) (FIG. 1A).

### Example 9-16: Chemical Composition of Antiscalants—Phosphorous Content of Antiscalants

The results of phosphorous content determination of antiscalants AS-1 to AS-8 are provided in FIG. 1B.

It was observed that AS-1, AS-2, AS-4 and AS-5 had phosphorous contents in the range of 60 to 100 μg P mg antiscalant-1. AS-3 and AS-7 showed a low phosphorous content, whereas AS-6 and AS-8 had a negligible phosphorous content. AS 8 had the lowest measured P content at 0.2 μg P mg antiscalant-1.

It was evident from FIGS. 1A and 1B that AS-1 to AS-5 and AS-7 had low carbon contents, whereas AS-6 and AS-8 had negligible phosphorous content.

### Example 17-24: Chemical Characterization of Antiscalants—NMR Spectra of Antiscalants

The ¹H, ¹³C, and ³¹P NMR spectra of AS-1 to AS-8 were recorded, and the results are shown in FIG. 1C.

It was observed from the NMR spectra that the ¹H, ¹³C, and ³¹P NMR spectra of AS-3, AS-4, and AS-5 were chemically highly similar. Antiscalants AS 6 and AS 8 were chemically the most complex and presented a multitude of peaks in their ¹H and ¹³C spectra. Except for AS 6, all antiscalants produced ³¹P NMR signals.

Hierarchical cluster analysis allowed the grouping of the antiscalants based on their chemical NMR fingerprint. The resulting dendrogram showed that AS-4 and AS-5 were the most similar and together with AS-3 they formed a cluster, and closer to this cluster was AS-2.

In contrast, AS-6 and AS-8 had a distinct chemical structure that separated them from the rest of the antiscalants (FIG. 1D).

Moreover, the chemical structure of each antiscalant was identified, or approximated, by comparing their NMR spectra to predicted spectra of common antiscalants.

The antiscalants AS-3, AS-4, AS-5 and AS-1 were identified as phosphonate-based (Table 1). The 1H peak at 3.3-3.7 ppm corresponds to H in the methylene group (—CH₂), the ¹³C peak at 72-57 ppm corresponds to the C in the N—C—P bond, and the ³¹P peak at 7-8 ppm is the C—P signal (FIG. 1C). Collectively these chemical shifts correspond to amino trimethylene phosphonic acid (ATMP); an antiscalant with three phosphonic acid groups.

AS-2 was also a phosphonate but with four phosphonic acid groups and was identified as hexamethylenediamine-tetra(methylenephosphonic acid) (HDTMP).

AS-7 was also found to be a phosphonate, but chemically different from the other phosphonate-based antiscalants. AS 7 was identified as 1-hydroxyethylidene-(1,1-diphosphonic acid) (HEDP). The ¹H peak at 1.4-1.5 ppm (—CH₃), the ¹³C peaks at 19 ppm (—CH₃) and 70 ppm (P—C—P), and the ³¹P peak at 19 ppm (P—C—P) are characteristic of the antiscalant HEDP.

In contrast, AS 6 and AS 8 were found to be polymers. AS-6 had 1H shifts at 3.5 and 4.5 ppm, which can be assigned to methine groups (=CH—) in hexose and pentoses, respectively. The 13C shifts at ca 60-70 and 75-80 ppm was assigned to —CH2 and =CH—, respectively. Furthermore, AS-6 had a typical carboxylic (—COOH) shift at 178 ppm in 13C spectrum (FIG. 1C). No ³¹P signal was detected for AS-6, thus this was the only phosphorus-free antiscalant (FIG. 1C). The NMR spectra of AS-6 resemble those of inulin—a carboxy polyfructose.

AS-8 spectra resembled those of the copolymer of maleic and acrylic acid (MA/AA), which is a common antiscalant. AS-8 contained other components besides MA/AA. For example, ³¹P shifts at 2 and 4.5 ppm indicated the presence of phosphate esters in trace amounts considering the low phosphorus contents (0.2 μg P mg antiscalant⁻¹) of AS-8.

The chemical characterization data and analysis of AS 1 to AS 8 is summarized as shown in Table 1.

TABLE 1
Antiscalant chemical characterization derived from datasheet and predicted NMR spectra
Antiscalant identification derived from datasheet and from predicted NMR spectra.

| | | Chemical | | |
|---|---|---|---|---|
| | | information | Identified from | Antiscalant |
| Antiscalant | Provider | in the datasheet | NMR spectra | type |
| AS 8 | A | polyacrylic acid and an | polyacrylate-maleic acid | polymer |
| | | acrylic acid copolymer | copolymer, with additives | |
| AS 6 | B | na^{a} | carboxy methyl inulin similar | biopolymer |
| AS 7 | C | Na | HEDP^{c} | phosphonate |
| AS 5 | B | Na | ATMP | phosphonate |
| AS 4 | A | ATMP^{b} | ATMP | phosphonate |
| AS 3 | C | Na | ATMP | phosphonate |
| AS 1 | D | ATMP | ATMP | phosphonate |
| AS 2 | D | ATMP | HDTMP^{d} | phosphonate |

| | | Detected | |
|---|---|---|---|
| Antiscalant | Color | odor | Chemical formula |
| AS 8 | pale yellow | none | (CH₂—CHCOOH)x- |
| | | | (HOOCCH═CHCOOH)y |
| AS 6 | amber to | none | C [symbol] H₁₁O₅-(C₅H₇O₇—CH₂—COOH) |
| | brown | | n-C [symbol] H [symbol] O₅—CH₂—COOH |
| AS 7 | none to pale | none | H₂O₃P—C(OH)(CH₃)-PO₃H₂ |
| | yellow | | |
| AS 5 | pale yellow | ammonia | |
| AS 4 | pale yellow | ammonia | |
| AS 3 | none to pale | none | N-(CH₂—H₂PO₃)₃ |
| | yellow | | |
| AS 1 | none to pale | ammonia | |
| | yellow | | |
| AS 2 | none to pale | benzoate | (H [symbol] PO₃—CH₂) [symbol] -N-(CH₂) [symbol] -N-(CH₂—H₂PO₃)₃ |
| | yellow | | |

^{a}na, not available.
^{b}ATMP, amino trimethylene phosphonic acid.
^{c}HEDP, 1-hydroxyethylidene (1,1-diphosphonic acid).
^{d}HDTMP, hexamethylenediamine-tetra(methylenephosphonic acid) [symbol]
indicates data missing or illegible when filed

### Example 25-32: The Bacterial Growth Potential of Antiscalants in Seawater with an Autochthonous Bacterial Population

To determine whether antiscalants would promote natural seawater bacterial growth, seawater with an initial bacterial cell concentration of 20,000 cells mL⁻¹ was incubated in the absence (reference control) and in the presence of 50 mg L⁻¹ of eight antiscalants. The bacterial growth of antiscalants was compared to that of the reference.

It was observed that the seawater microorganisms respond to the presence of antiscalants. The phosphonate-based antiscalants AS-1 and AS-2 resulted in the highest bacterial growth.

The reference bacterial growth in the seawater without added antiscalant reached about 1.5×10⁶ cells mL⁻¹ after 8 days of incubation.

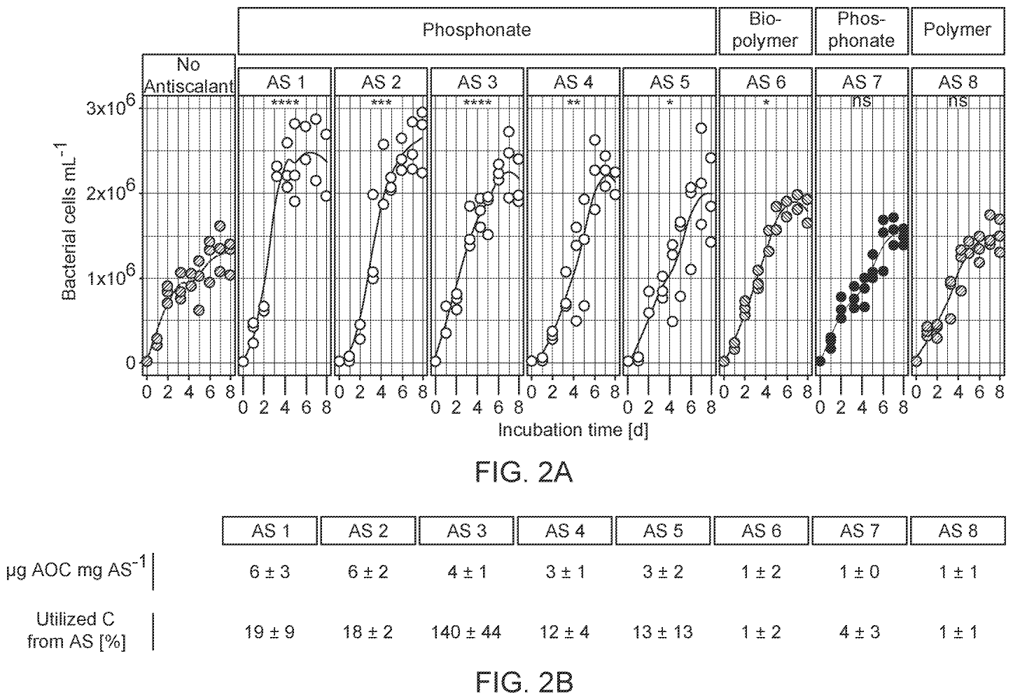

In contrast, when phosphonate-based ATMP (AS-1, AS-3, AS-4, and AS-5) and HDTMP (AS-2) antiscalants were added, the bacterial growth curves significantly differed from that of the reference (p<0.05, one-tail Wilcoxon signed-rank test) with cell numbers reaching about 2×10⁶ to 3×10⁶ cells mL⁻¹ (FIG. 2A).

The biopolymeric antiscalant AS-6 slightly promoted microbial growth.

Distinctively, the antiscalants similar to the phosphonate HEDP (AS-7) and the synthetic polymer AA-MA (AS-8) did not promote growth (p>0.05), and their resulting bacterial growth resembled that of the reference (FIG. 2A).

The results are summarized in FIG. 2A. FIG. 2A shows the bacterial growth potential of different antiscalants (AS) in Red Sea water. All phosphonate-based antiscalants, except AS 7, promoted higher bacterial growth than the control with no antiscalant. The growth potential curves of antiscalants AS7 and the polymer AS 8 were similar to that of the control. Data of three biological replicates is shown. Significance levels: **** p<0.0001, *** p<0.001, **p<0.01, * p<0.05, ns=not significant; one-side Wilcox test compared to the control.

The number of bacteria that grew in the presence of an antiscalant in excess to that of the control (without antiscalant) can be expressed in terms of carbon utilized from antiscalant to produce such number of bacteria. Comparing those values with the measured TOC in antiscalants may inform on the antiscalants' biodegradability.

To this end, we considered the net number of cells produced after 8 days of incubation (FIG. 2A), and a yield for seawater bacteria of 4.6×10⁶ cells μg C⁻¹ reported by (Dhakal et al., 2021). The results were expressed in terms of assimilable organic carbon from the antiscalant; g AOC mg antiscalant⁻¹. FIG. 2B shows the bacterial growth from antiscalant expressed as assimilable organic carbon (AOC) was calculated assuming a yield of 4.6×10⁶ cells μg C⁻¹. An estimate of antiscalants' biodegradability (theoretically utilized C) is based on their AOC and TOC ratios.

It was observed that the polymeric antiscalants (AS-6 and AS-8), and the HEDP-like phosphonate (AS-7) were hardly biodegradable. For example, the estimated AOC from AS-8, under the tested conditions, was about 1 μg AOC mg antiscalant⁻¹, which represents a biodegradability of 1% for this antiscalant (FIG. 2B).

In contrast, the AOC derived from the growth potential on all phosphonates ATMP-like antiscalants ranged from 3 to 6 μg AOC mg antiscalant¹. In particular, the biodegradability of AS-3 derived from its growth potential was above (by about 40%) the measured TOC content for this antiscalant (FIG. 2B). Thus, indicating that phosphorus in the antiscalant promoted bacterial growth and that the growth of microorganisms in the seawater during the growth potential test was phosphorus limited.

What is claimed:

1. A method for determining the microbial growth potential of at least one antiscalant composition in natural seawater comprising at least the following steps:
filtering natural seawater to obtain seawater comprising bacteria having a predetermined bacterial cell concentration;
adding at least one antiscalant composition comprising at least one antiscalant to the seawater to obtain a mixture, wherein the concentration of the at least one antiscalant is in the range of 0.1 to 100 mg/L; and
incubating the mixture, and determining the bacterial cell concentration of the mixture at regular intervals to determine the microbial growth potential of the at least one antiscalant composition in the natural seawater.

2. A method according to claim 1, wherein the bacteria are autochthonous seawater bacteria.

3. A method according to claim 1, wherein the predetermined bacterial cell concentration is in the range of 5000 to 50000 bacterial cells mL⁻¹.

4. A method according to claim 1, wherein the weight of the at least one antiscalant is in the range of 50.0 to 99.0 wt. % based on the weight of at least one antiscalant composition.

5. A method according to claim 1, wherein the at least one antiscalant is selected from phosphonate-based antiscalant, polymer-based antiscalant, and biopolymer-based antiscalant.

6. A method according to claim 1, wherein the at least one antiscalant is selected from amino trimethylene phosphonic acid, 1-hydroxyethylidene-(1,1-diphosphonic acid), hexamethylenediamine-tetra(methylenephosphonic acid), polyacrylate-maleic acid copolymer, and carboxy methyl inulin.

7. A method according to claim 1, wherein, the concentration of the at least one antiscalant in the mixture is in the range of 0.1 to 100 mg/L.

8. A method according to claim 1, wherein the mixture is incubated at 20° C. to 40° C.

9. A method according to claim 1, wherein the mixture is incubated in dark.

10. A method according to claim 1, wherein the mixture is incubated for 2 to 10 days.

11. A method according to claim 1, wherein the bacterial cell concentration of the mixture is determined at 24-hour intervals.

12. A method according to claim 1, wherein the bacterial cell concentration of the mixture is determined by flow cytometry.

13. A method according to claim 1 further comprising a step of determining the chemical structure of the at least one antiscalant present in the at least one antiscalant composition.

14. A method according to claim 1, wherein the chemical structure of the at least one antiscalant is determined based on at least one technique selected from ¹H, ¹³C, and ³¹P NMR spectra.
